(19) United States
(12) Patent Application Publication
(10) Pub. No.: US 2006/0229488 A1
(43) Pub. Date: Oct. 12, 2006

(54) Blood pressure detecting device and system

(76) Inventors: Peter Joseph Ayre, Crows Nest, NSW (AU); Geoff Douglas Tansley, Mt. Colah, NSW (AU)

Correspondence Address: McCarter & English;Angelica Brooks, City Place I, 185 Asylum Street, Hartford, CT, 06103, US

(21) Appl. No.: 10/565,056

(22) PCT Filed: Jun. 24, 2004
(86) PCT No.: PCT/AU04/00829

§ 371 Date: May 22, 2006

(30) Foreign Application Priority Data
Jul. 18, 2003 (AU) 2003903726

(51) Int. Cl. A61N 1/362; A61B 5/02

(52) U.S. Cl. 600/17; 600/485; 600/486

(57) ABSTRACT

An implantable device including a cuff (8) positioned to contact the outer surface of a tubular body (9) carrying blood. At sensor (13) to measures blood pressure is encapsulated within the cuff. The device does not occlude or adversely affect the flow or blood pressure within a patient's circulatory system. Also disclosed is a system to control an implantable blood pump using a pressure based on a pressure measurement of blood flow at the inlet to the pump.

### FIELD OF INVENTION

The present invention relates to an implantable device and a system for detecting blood pressure and/or pumping state of a patient's circulatory system for use with a blood pump.

### BACKGROUND

Congestive Heart Failure (‘CHF’) is a disease of great importance. CHF typically results in a deterioration of heart function. A common feature of CHF is that it results in impairment of the performance of the heart's pumping action.

Previously, it has been suggested that the symptoms of CHF can be at least addressed by the use of Left Ventricle Assist Devices (‘LVADs’) which assist the heart's normal function and reduce the overall pumping load on the heart.

These LVADs typically pump blood from the left ventricle of a heart to a distal region of the circulatory system usually the ascending aorta. One of the main problems associated with the use of LVADs is that over-pumping or under-pumping adversely affects the valves of the heart.

The result of over-pumping or under-pumping is that it places undue stress on the valves and may break or become a site for thrombogenesis. These events may even lead to further deterioration of the health of a patient and in most extreme cases, may lead to the death of a patient from stroke or formation of blood clots in the circulatory system.

Current controllers which assist in the control of LVADs and related blood pumping devices rely on various sensors to provide information.

Sensors measuring blood flow and pressure have been used for control in this context and have been placed in contact with the blood stream thereby presenting a site for thrombogenesis.

The reliability of these sensors may be a problem as these sensors may fail because they measure blood flow or pressure invasively within the circulatory system of a patient. “Invasively” for purposes of this specification means that the device, in use, directly contacts the blood of the patient.

As a result, there has been a long felt need for an invention that non-invasively detects arterial blood pressure, which is suitable for use in cooperation with a blood pumping device or system.

Previously, U.S. Pat. No. 5,289,821 (Swartz et al) and U.S. Pat. No. 6,398,734 (Cimochowski et al) describe a cuff device for measuring only blood flow rates. These blood flow rates do not in all circumstances allow detection for the estimation of the pumping state of the heart. Additionally, U.S. Pat. No. 5,289,821 includes a sensor which is capable being removed from the cuff and this may lead to problems of accidental disconnection of the sensors.

Also, Japanese Patent Publication No. 2002-224006 (Kinchi et al) describes a system wherein the blood flow is detected and the blood pressure is estimated from the blood flow rate by an arithmetic unit. This system only outputs an estimated value of blood pressure and fails to detect the real value of blood pressure. Additionally, the output is delayed and this means that the data cannot be directly used for real time applications, such as to cooperate as a feedback mechanism for a speed control of a blood pump.

Also, there are many known methods and devices capable of providing a control system for an implantable blood pump, which may assist or replace the operation of a patient's heart. These implantable blood pumps generally operate at a constant speed set-point and are unresponsive to changes in the physiological condition or natural pumping state of a patient. Thereby, the blood pump may be under-pumping or over-pumping.

U.S. Pat. No. 5,385,581 (Bramm et al) and U.S. Pat. No. 6,623,420 (Reich et al) describe similar methods to overcome the problem with the inclusion of pressure sensor(s) in the inlet of the blood pump. The output of said pressure sensor(s) is then fed back into the control system of the blood pump. The blood pump's speed is then adjusted according to a comparison of the current inlet pressure against the desired inlet pressure. These systems fail to take into account that a patient's desirable inlet pressure changes due to physiological conditions, and that only the minimum pressure over time can reliably predict under pumping or over pumping of an implantable blood pump. Additionally, U.S. Pat. No. 6,623,420 assumes the flow rate is constant and has a mean value, which is not physiologically accurate.

Previously, other types of systems have been used to control and adjust the speed of implantable blood pumps. U.S. Pat. No. 6,227,797 (Watterson et al) describes a system of using back EMF generated by the motor of the implantable blood pump to detect rotor location. This rotor location may then be used to determine the speed of rotation of the pump impeller. The controller then may calculate an estimated flow rate of blood through the pump, based on the detected speed of rotation. The estimated flow rate may be used in a closed loop feedback system. This closed loop feedback system adjusts the pumping speed of the pump to correct the difference between desired flow rate and the estimated flow rate. Flow rate, in isolation, is not suitable to be used as a feedback parameter to detect under-pumping. The flow rate does not allow the controller to determine the perfusion rate of the pump.

It is an object of the present invention to address or ameliorate at least one of the above disadvantages.

### BRIEF DESCRIPTION OF THE INVENTION

According to a first aspect of the present invention consists in an implantable device, including: a cuff positioned to contact the outer surface of a tubular body carrying blood; and at least one sensor which measures blood pressure encapsulated within said cuff, wherein said cuff is integrally formed within a cannula.

Preferably, said device does not occlude or adversely affect the flow of blood or blood pressure within a patient's circulatory system.

Preferably, said device includes at least two sensors and said sensors are aligned axially in respect to said tubular body.

Preferably, said device includes at least two sensors and said sensors are aligned radially in respect to said tubular body.

Preferably, said device is connected to a controller that determines the pumping state of said heart from changes in said pressure.

Preferably, said cuff comprises: silicone, velour or Dacron™.

Preferably, said device cooperates with a blood pump.

Preferably, the blood pressure is used in a feed back mechanism having a controller to control the pumping speed of said blood pump, said feed back mechanism including a controller.

Preferably, said controller adjusts pumping speed to minimise under-pumping and over-pumping by the implantable blood pump.

### BRIEF DESCRIPTION OF THE DRAWINGS

Embodiments of the present invention will now be described with reference to the accompanying drawings wherein:

FIG. 1 is a schematic view of a first preferred embodiment of an implantable device implanted within a patient;

FIG. 2 is a perspective and enlarged view of a portion of the implantable device shown in FIG. 1;

FIG. 3 is a schematic view of a further embodiment cooperating with a blood pumping system; and

FIG. 4 is a graph demonstrating pumping states of a heart (one cardiac cycle);

FIG. 5 is a diagram of a further embodiment of the present invention;

FIG. 6 is a cross-sectional side view of a portion of the embodiment shown in FIG. 5;

FIG. 7 is a graph showing the actual blood pressures within the inlet of a further embodiment of the present invention over time; and

FIG. 8 is a graph showing the preferred detected pressure of a further embodiment of the present invention.

### BRIEF DESCRIPTION OF THE PREFERRED EMBODIMENTS

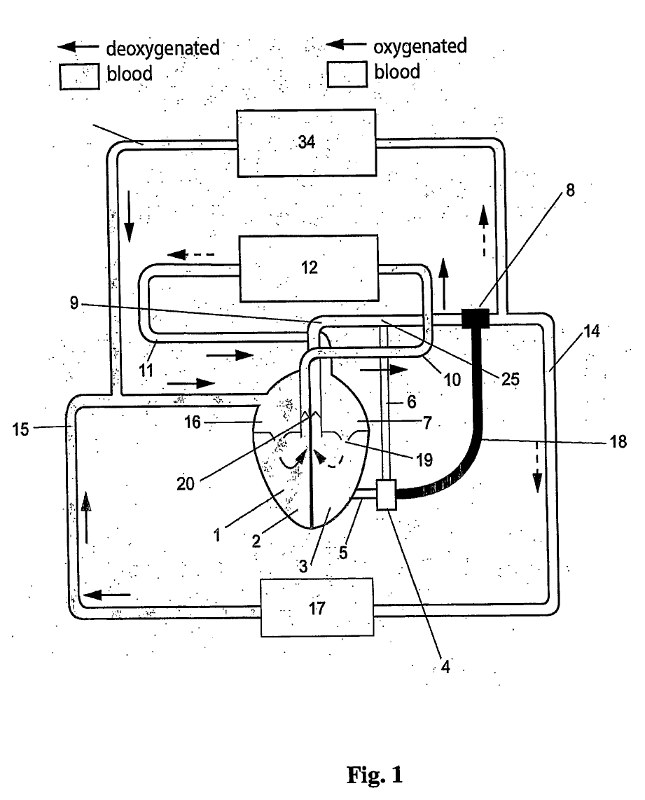

A first embodiment of the present invention is shown in FIG. 1 and shows schematically a portion of a circulatory system of a patient. In this embodiment, the arteries function as tubular bodies containing blood. FIG. 1 additionally shows a blood pump 4, in situ, and blood pump 4 may be implantable and suitable for use as a Left Ventricle Assist Device (‘LVAD’). A heart 1 pumps blood from pulmonary vein 11 into aorta 9 via the left atrium & left ventricle 3. The left atrium 7 receives blood from the pulmonary veins 11 and this blood flows into the left ventricle 3. In diseases, such as congestive heart failure, the left ventricle 3 may fail or poorly pump blood. Previously, it has been suggested that left ventricular failure may be treated with the use of an implantable pump such as blood pump 4. Preferably, blood pump 4 may be a VentrAssist™ LVAD. The description of this device is contained within U.S. Pat. No. 6,227,797 and forms part of this description.

LVADs preferably require a detection mechanism to detect the physiological condition of the patient and the pumping state of the heart 1. This detection mechanism preferably feeds back information and data to the controller mechanism (not shown) of the blood pump 4. The controller mechanism (not shown) may then adjust the pumping rate or speed as required. Implantable pumping systems often may interfere with a patient's normal pulsatile blood flow. Some patients may experience continuous arterial blood flow rather than pulsatile arterial blood flow as a result of the pumping system and this may interfere with the normal operation of the valves of the heart. If the valves are permanently open or closed, blood clots may form around these regions of the circulatory system.

It is preferable for a ventricle to eject blood through all four of the heart valves 19 & 20 when a Ventricle Assist Device (‘VAD’) is present. This may reduce the risk of clot and other serious complications. The particular pumping state resulting from all four other said valves ejecting may generate an arterial pulse.

Oxygenated blood flows from the left atrium 7 of the heart 1 into the left ventricle 3 where the blood is pumped into the aorta 9. The aorta 9 connects to arterial system 14. Thereby oxygenated blood is delivered to the entire body, which includes brain/head regions 34 and lower distal regions 17 such as the legs, by relying on blood pumping pressure supplied by the left ventricle 3.

The oxygenated blood is then utilised by the brain/head regions 34 and lower distal regions 17. The deoxygenated blood is then delivered to the venous system 15. The deoxygenated blood then travels along the venous system 15 to the right atrium 16 of the heart 1. The right ventricle 2 pumps deoxygenated blood into the pulmonary artery 10. The blood then travels to the lungs 12 where it is re-oxygenated. The oxygenated blood then returns to the left atrium 7 of the heart 1 via the pulmonary vein 11.

A blood pump 4 is connected to the apex of the left ventricle 3 by way of an inflow cannula 5. The blood pump 4 pumps blood into the outflow cannula 6 and this outflow cannula 6 delivers the blood to the aorta 9.

This embodiment provides a non-invasive means of detecting the pumping state of the heart and the positions and/or movement of the various heart valves. Furthermore, the pumping state information or blood pressure measurements may be used in a feedback mechanism to the pumping speed of blood pump 4.

In the embodiment featured in FIGS. 1 & 2, a patient's circulatory system has been implanted with blood pump 4. This blood pump 4 preferably assists the left ventricle 3 to pump blood into the arteries such as the aorta 9. The blood pump 4 is connected to the apex of the left ventricle 3 by stenting or cannulation using an inflow cannula 5. This inflow cannula 5 provides blood from the left ventricle 3 to the blood pump 4. The blood pump 4 preferably pumps blood to the aorta 9 which is downstream of the left ventricle 3. The blood pump 4 delivers blood to a position 25 by way of an outflow cannula 6. The blood pump 4 is powered and controlled by a percutaneous lead (not shown) which connects to an external pump controller (not shown) and an external power supply (not shown).

The percutaneous lead 5 also supplies the pump 4 with a means of two way data flow to the pump controller. The pumping speed of the blood pump 4 is controlled by the pump controller. Preferably, the blood pump 4 includes sensors 13 which send information to the pump controller by internal wiring 18 and the pump controller uses this information to adjust the pumping speed appropriately.

In FIGS. 1 & 2, a cuff 8 is preferably positioned around a portion of the aorta 9 and this portion may be downstream of position 25. The cuff 8 may be secured to the artery by: stitching; bioglue; or by encouraging the patient's body to incorporate the cuff 8 and thereby embed it within an outer surface of said artery or aorta 9. The cuff 8 may be constructed of the following materials: velour, silicone, polyetheretherketone (‘PEEK’), polyurethane, polymer and/or graft material. Please note that the cuff 8 may be constructed of various other biocompatible materials.

The cuff 8, which is a thin walled substantially tubular member, includes at least one non-invasive pressure sensor 13, which is preferably encapsulated within the cuff 8. Sensors 13 may detect blood pressure within the aorta 9 without directly contacting the blood as sensors 13 may be constructed of relatively bio-toxic materials. The encapsulation of sensors 13 minimises the risk of serious complications to the patient in respect of infection and possible bio-toxic leakage of the sensor.

Detection of adverse pumping conditions (eg. ventricular suction, fluid flow modulation and/or fault conditions) affecting a patient's heart may be achieved through analysis of signals produced by the non-invasive pressure sensors 13. The sensors 13 may use: acoustic sensors (eg microphone); vibration sensors (eg piezo-electric sensors); and/or Micro-Electro-Mechanical Systems (‘MEMS’) based technology, which may be preferably permanently embedded within the cuff 8. Electrical signals generated by the sensors 13 are sent to the pump controller (not shown) where by analysis of this signals can yield a pumping state of the heart and determine the appropriate pumping speed. Additionally, the sensors 13 may be manufactured of a piezoelectric material that generates an electric signal then the material is distorted in shape. This piezoelectric material may include specialised polymers.

In other embodiments, the cuff 8 may be attached to other tubular bodies containing blood including arteries, veins, stents and cannulae. The cuff 8 may be attached to the pulmonary vein 11 for detection of suction events which may be caused by excessive drain of blood from said pulmonary vein 11. This drain may be caused by a blood pump 4 connected in a similar configuration as that of blood pump 4. In situations where blood pump 4 pumps excessive amounts of blood from the left ventricle 3, the aortic valve 20 may remain closed and prevent normal blood circulation into the aorta 9 between location 25 and the aortic valve 20. If the overpumping of blood pump 4 is increased, this suction event may lead to ventricular collapse of the left ventricle 3. The suction event may also lead to mitral valve 19 being continuously open as the blood would be drawn directly from the pulmonary vein 10 into the left atrium past the mitral valve 19 into the left ventricle 3. This may result in a lack of blood pulsatility and thrombogenesis may occur. Overpumping events are also not desirable and should be avoided.

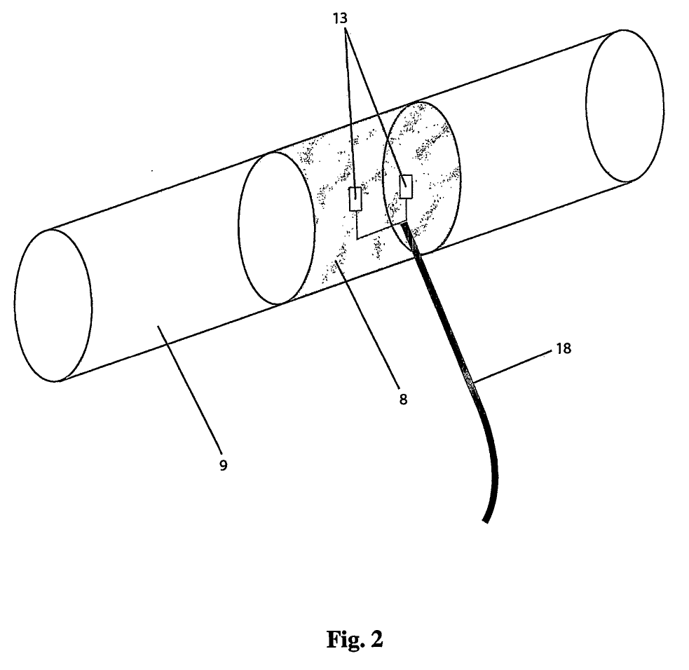

In FIG. 2, the cuff 8 surrounds the outer surface of aorta 9. The two sensors 13 are axially disposed along the length of the cuff 8 and are preferably joined within the cuff 8. The axial aligned sensors 13 may measure blood flow or pressure at a position close to where the inner wall of cuff 8 contacts the outer wall of the aorta 9. Preferably, the axially aligned sensors 13 may provide a differential pressure measurement along the length of the cuff 8 or alternately provide for additional sensor redundancy.

It is desirable to use pressure sensors 13 to determine the actual blood pressure. In the prior art, calculated or estimated values of blood pressure derived from actual measurement of blood flow often fail to compensate for the characteristics of blood as a liquid (namely blood may be of variable compressibility and/or viscosity).

In an alternative not shown embodiment, it may also be desirable to position sensors 13 at radial intervals around the cuff 8. These radially aligned sensors 13 may differentially detect different pressures or flows experienced by the sensors. This information may be used to calculate an average pressure relative to an axial section of the cuff 8 or may allow for sensor redundancy in cases of device failure.

This embodiment may be modified so as to allow the cuff 8 to be positioned on or around the inflow cannula 5 rather than a portion of the aorta 9. This would allow detection of blood flow and/or pressure into the blood pump 4. The resolution of the pumping states of the heart 1 may be increased by the sensors 13 due to the proximity of the heart 1 and that the sensors 13 are positioned upstream from the blood pump 4, which preferably generates a continuous blood flow and tends to override the normal pulsatile blood flow of the patient. Alternately, the cuff 8 may be integrally moulded within the body portion of the inflow cannula.

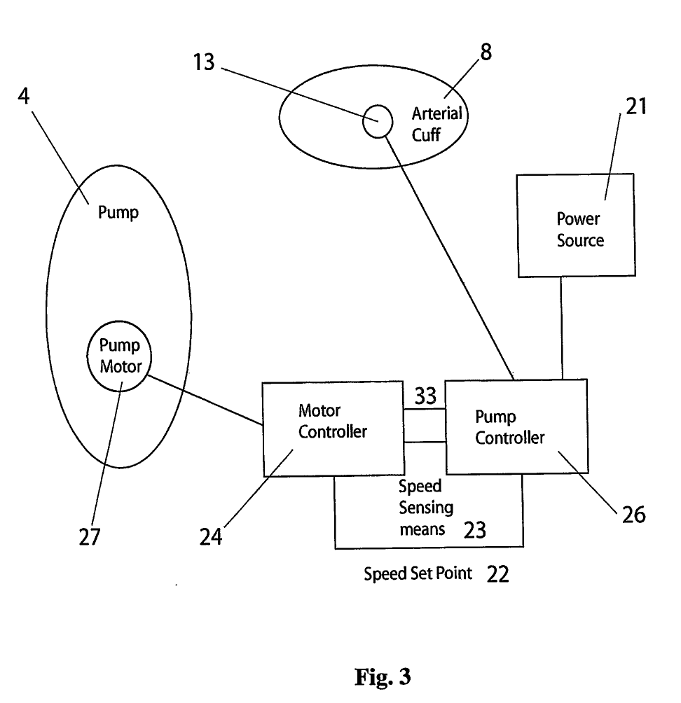

According to a further embodiment, shown in FIG. 3, a pump controller 26 is supplied with power by a power source 21. This power source 21 may include batteries or mains power. The pump controller 26 may also receive input data and information from the motor controller 24 in the forms of a power sensing means 33 and speed sensing means 23 and electric signals from the sensors 13. The pump controller 26 may then calculate an appropriate pumping state and/or speed. The pump controller 26 then issues a speed set point 22 to the motor controller 24. The motor controller 24 controls the actuations of the pump motor 27 located within the blood pump 4.

All of the described embodiments of the present invention may be easily modified for use with Right Ventricle Assist Devices (‘RVADs’) or other types of blood pumps.

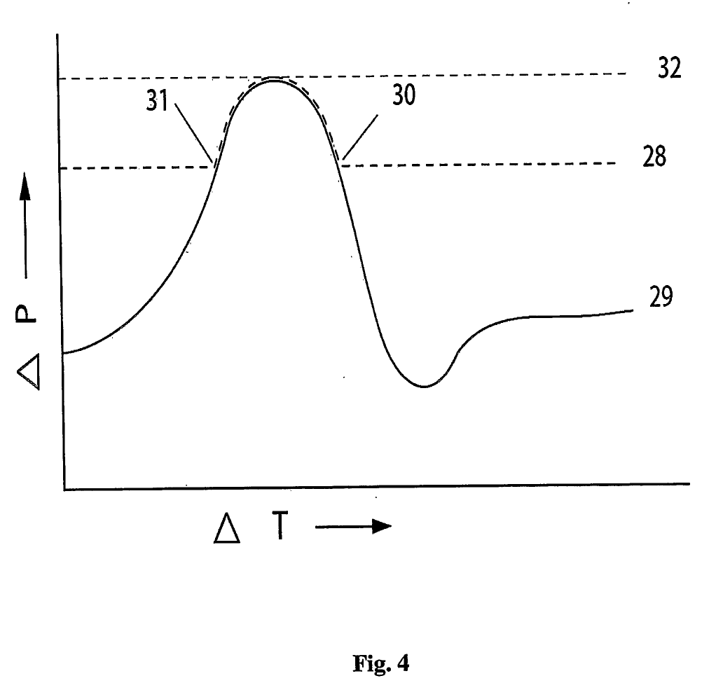

FIG. 4 shows the various cardiac pressure outputs plotted against time as measured within the aortic artery. A normal cardiac pressure output is shown by graph line 29. Graph line 29 demonstrates a typical person's pressure output; please note that this person does not have an implantable continuous flow LVAD or blood pump 4. Graph line 28 graphically displays the pressure output of a similar person, as shown in graph line 29, wherein a continuous flow LVAD is implanted and is actively assisting the heart. Position 31 shows the point at which the aortic valve opens and position 30 shows the position at which the aortic valve of the patient's heart closes. It can be seen that the LVAD raises the baseline pressure within the artery and thereby reduces the pulsatility of the patient's circulatory system. The reduction of pulsatility may lead to problems in externally detecting the patient's condition in the traditional ways.

In a further situation, a similar patient, to the one displayed in graph line 29, is implanted with a continuous flow LVAD and the LVAD is pumping at a higher pressure than the pumping pressure of the heart. Thereby the aortic valve 20 is not opening and closing and the pulsatility is completely removed. In this situation, the abovementioned embodiment may be able to detect blood flow and pressure rates whereas tradition methods would fail to detect the pumping state of the patient.

In the abovementioned embodiment, the blood pressure information may then be utilised to determine the cardiac pumping state of the patient. These states may include: Total Ventricular Collapse (‘TVC’) and Pump Regurgitation (‘PR’), which produce low flow through the blood pump 4. TVC state produces non-pulsatile low flow while PR produces pulsatile low flow less than 1 L/min. States such as Partial Ventricular Collapse (‘PVC’), Aortic Valve Closed (‘AC’) and Ventricle Ejecting (‘VE’) produce normal pump flows greater than 1 L/min. PVC and PR states can be differentiated from AC state since flow pulsatility is more evident. PVC state can be differentiated from VE state as the dynamic flow profile is different from all other states. The dynamic nature of the blood flow is reflected by intravascular blood pressure and/or intravascular blood flow and it is this that is detectable by sensors 13.

In examining in-vitro and in-vivo data, it has been found that TVC state may be detected by a fall of pump flow to near 0 L/min accompanied by a reduction of flow pulsatility, which is detectable by sensors 13.

The PVC state is indicated by a variation in profile of the instantaneous pump speed waveform given a level of pulsatility derived from the sensor(s) 13. Given that normal flow rates can still be observed during this state and that flow pulsatility is large, the only parameter distinguishing this state from the VE state is the flow profile, which may also be detectable by the sensor(s) 13.

By analysing the cardiac cycle with the pump it has been found that there may be a portion of AC state where the aortic valve remains closed, whilst however the pump flow is still pulsatile. This portion defines a point beyond which pump flow pulsatility may be reduced. At high perfusion demands, as in exercise, the failed ventricle may be supplemented to such an extent that the flow through the blood pump 4 is preferably pulseless. If no left ventricle contraction occurs then implantable rotary blood pump flow will be non pulsatile. Contraction of the left ventricle 3 with the blood pump 4 connected means that pump head is proportional to the difference between the aortic pressure and the Left Ventricular Pressure (‘LVP’). If the work of blood pump 4 is increased beyond the point that the left ventricle 3 is doing no work (the aortic valve 20 no longer opens) maximum LVP begins to decrease. The minimum instantaneous pump differential pressure will begin to rise relative to the RMS of the pump differential pressure over the cardiac cycle. If the left ventricle 3 is weakened through heart failure this will occur at relatively lower pump speeds and the mitral valve will still continue to open and LVP maximum will decrease towards zero with increasing speeds. Steady flow occurs when there is no pulsatility in the speed signal and the mitral valve never closes. The target speed at which this occurs will increase with SVR or VR and cardiac contractility. Continuing to increase the pumping speed of blood pump 4, may further the transition from pulsatile to non pulsatile blood flow. The detection of the VE state and AC state can only be achieved dynamically by considering the maximum instantaneous speed Nmax (t) and the rms of instantaneous speed Nrms(t) for the nth and (n−1)th cardiac cycle. A significant change occurs only if there is a change in average pump speed set point, after load or pre-load. A method of detecting the AC state without relying on transitions has been chosen which uses peak to peak flow rate that pump flow is greater than 1 L/min.

The VE state may be identified non-invasively by pump flow rate being larger than 1 L/min and peak to peak instantaneous voltage (flow) being greater than a threshold value and the flow symmetry being greater than that for the PVC state.

The PR state may be indicated when the pump flow falls below the lower flow limits Qmin which is set to be 1 L/min. This level of Qmin is set at 1 L/min although not “0 L/min” may be considered a safe limit to be classed as retrograde flow.

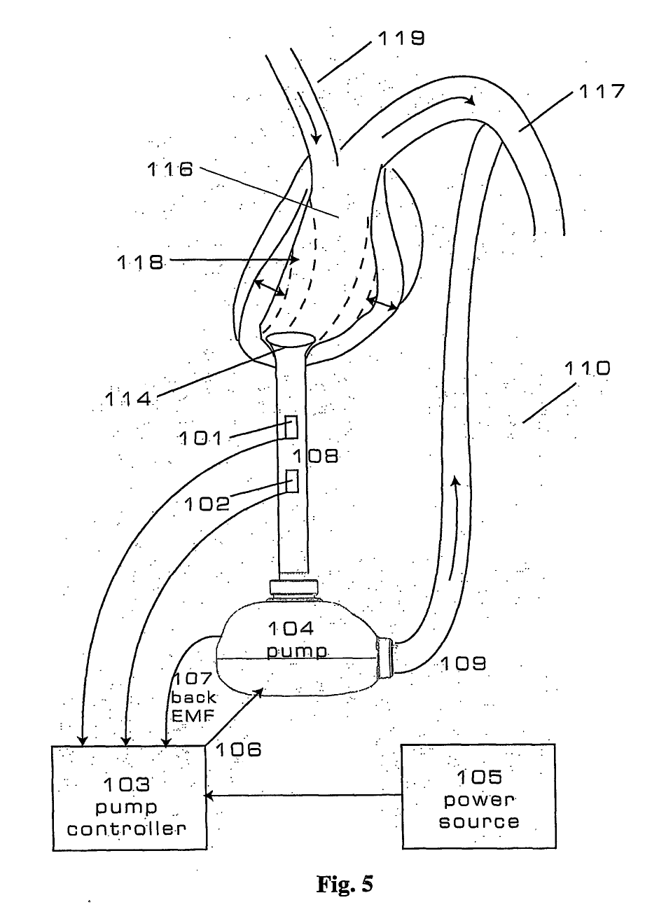

According to a further embodiment of the present invention as depicted in FIG. 5, the present invention may include a system 110. Preferably, this system 110 includes an implantable blood pump 104 in parallel fluid communication between the apex of the left ventricle 116 of a patient and the aorta 117. This implantable blood pump 104 functions to pump blood from the left ventricle 116 along the inflow cannula 108 through the pump 104 and then down the outflow cannula 109 into the aorta 117. The implantable blood pump 104 may be of centrifugal rotary assist device as described within U.S. Pat. No. 6,227,797.

The implantable blood pump 104 is controlled by a controller 103. The controller 103 is supplied with power from a power source 105 and this power is then used to drive the implantable blood pump 104. The controller 103 specifically sets a speed set-point for the implantable blood pump to operate at. Preferably, the controller 103 adjusts the speed set-point in accordance with the most desirable pumping state of the natural heart.

The desired pumping state may be determined by the use of sensors integrally moulded within the inflow cannula 108. Blood pressure sensors 101 and blood flow sensors 102 may be encapsulated within a cuff and wherein said cuff is embedded within the inflow cannula 108. Both the pressure sensors 101 and blood flow sensors 102 provided data to the controller 103.

The pressure sensors 101 and blood flow sensors 102 preferably measure blood flow rates and pressures within the inflow cannula 108. The preferred location for the sensors (shown in FIGS. 5 & 6) is proximal to the inflow cannula 108 or the inlet of the implantable blood pump 104 because pressures and flow rates are substantially more difficult to accurately measure in respect of the outflow cannula 109.

The pump 104 may also supply data and/or information to the controller relating to back EMF generated by the movements of an impeller within the pump body. This back EMF supplies may supply information specifically pertaining to the instantaneous position of the impeller and the controller 103 may use this information to determine the rate of rotation of the impeller and may then extrapolate an estimated value for blood flow through the blood.

In the embodiment shown in FIG. 5, the controller 103 uses the detected pressure (from the pressure sensors 101) and the estimated blood flow rate (derived from the back EMF generated by the implantable blood pump 104) to determine a current pumping state of the heart or left ventricle 116.

The system 110 preferably allows the controller 103 to detect whether under-pumping or over-pumping of the left ventricle 116 has or is occurring.

Over-pumping of the left ventricle 101 occurs when the implantable blood pump 104 is pumping too much blood. In this situation, the septum and inner walls of the left ventricle 116 move to position 118. The resultant action is called ‘suck-down’ of the left ventricle 116. Overpumping may lead to low blood flow rates due the partial or full collapse of the inner walls and septum of the left ventricle 116. This collapse may occlude the inflow cannula 108 and may also block the operation of the patient's aortic valve (not shown).

Under-pumping occurs when in sufficient blood is being pumped by the implantable blood pump 104. The result is that there is insufficient filling of the left ventricle 116 and this may lead to a “damming” effect in the left atrium or pulmonary vein (shown in FIG. 5 as 119). In the worst cases, excessive blood may build up in the lungs of the patient (not shown). This damming effect is not unlike symptoms seen in relation to right ventricle failure patients. Obviously, under-pumping should then be avoided.

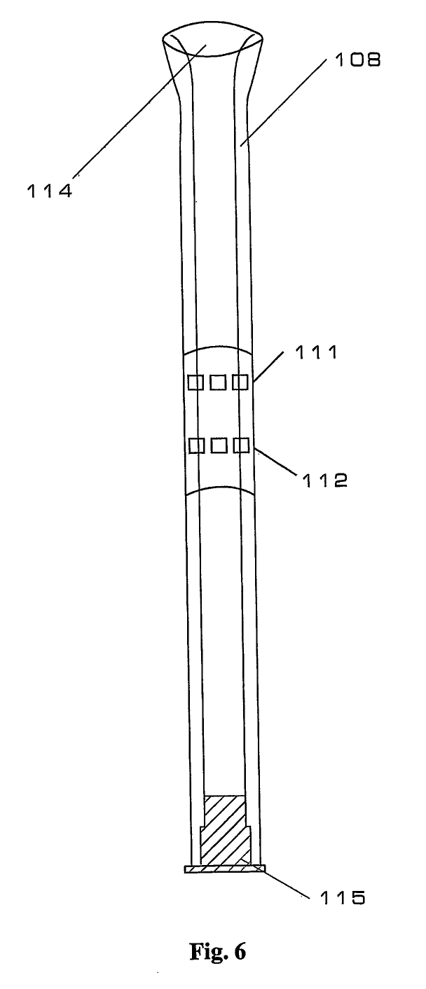

FIG. 6 shows an enlarged view of a portion of the system 110, in which an inflow cannula 108 is shown. This inflow cannula 108 includes a funnel shaped tip 114, which is preferably inserted within a cored hole of the apex of the left ventricle 116. The inflow cannula 108 forms a blood conduit between the left ventricle 116 and an implantable blood pump 104. When in use, the implantable blood pump 104 screwably attaches to the pump connector 115.

The inflow cannula 108, in FIG. 6, may include two sets of sensors: first set 111 and a second set 112 of pressure sensors. Preferably, these sets of sensors 111 & 112 are encapsulated within a cuff which is embedded within the walls of the inflow cannula 108. The walls and funnel tipped end 114 of the inflow cannula 108 may be constructed of biocompatible material such as silicone.

Each of sets of sensors 111 & 112 comprise multiple radially dispersed sensors. This radial dispersion may allow the controller system to find an average value pressure at an axial position. This averaging of sensor reading at various axial locations allows the controller 3 to compensate if the cannula kinks or bends. Generally, the bending or kinking of the inflow cannula 108 may occur during implantation and may induce variable pressures to occur at various axial cross sections. Additionally, the radially dispersed sets of sensors may allow the system 110 to have inbuilt redundancy in case of single sensor failure.

The sets of sensors 111 & 112 are also preferably axially spaced apart in relation to each set. The axial dispersion of the sets 111 & 112 may allow for differential readings of pressure and flow to be taken at various axial intervals along the length of the inflow cannula 108. The differential pressure readings between sets of sensors 111 & 112 along the axial length of the inflow cannula 108 may be used by the controller 103 to determine blood flow rate without the need for additional sensors.

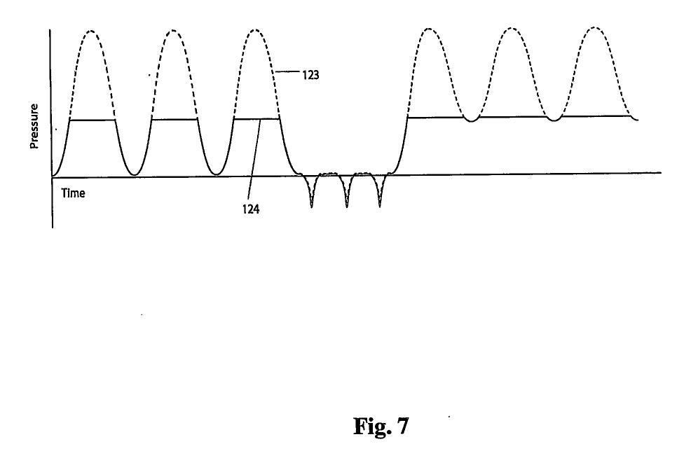

FIG. 7 shows a graph of an example blood pressure experienced by the patient's blood within the inflow cannula 108 over time. The optimal or desired pressures are demonstrated by a first region 120. This first region 120 shows three cardiac cycles of a patient where the blood pressure is pulsing between 0 mmHg and up to 200 mmHg (more generally the upper range is approximately 120 mmHg).

The second region 121 shows the blood pressures experienced within the inflow cannula 108 during overpumping or a “suck down” event over three cardiac cycles. The maximum pressure during this second region is relatively low or close to 0 mmHg and thereby flow is greatly reduced during overpumping of the left ventricle 116. The minimum pressure is generally −20 mmHg. However, it is common to see only relatively small negative peaks varying between −1 mmHg to −20 mmHg.

The third region 122 shows the blood pressures experienced within the inflow cannula 108 during under-pumping over three cardiac cycles. Typically, the maximum pressures experienced are comparable to the first region 120. However the minimum pressure baseline is increased from 0 to approximately 10 mmHg. The pumping state of under-pumping may be difficult to detect using blood flow rate sensors, only. Because the flow rate in the inflow cannula 108 is comparable to the first region 121 and thereby not allowing the physician to successfully diagnosis the differences between under-pumping and correct pumping.

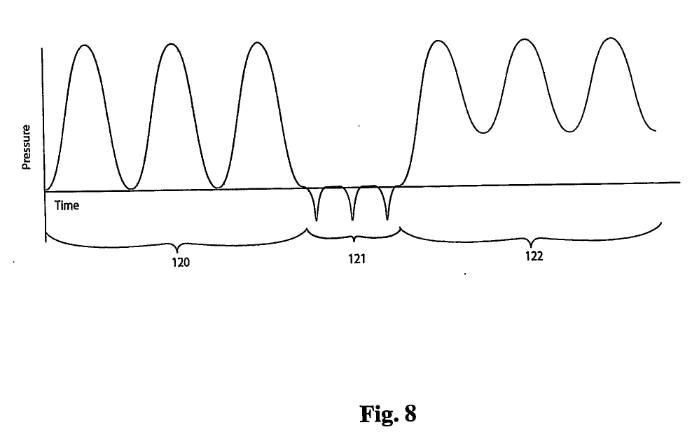

In FIG. 8, another graph is shown. This graph denotes the actual blood pressure of a patient over time by the use of a dotted line 123. The dotted line 123 matches the graph of FIG. 8 and supplied for comparison purposes. The full line 124 shows the pressure values detected by the sets of pressure sensors 111 & 112.

The full line 124 shows that the output of blood pressure measurement only between certain predetermined ranges. Conventional pressure sensors available for commercial application typically only detect specific pressure ranges. The pressure ranges shown are generally between −50 mmHg and +200 mmHg. Conventional pressure sensors used for implantation cannot accurately and precisely determine the pressure over such broad ranges to the level of accuracy necessary for this application. However, if the range of blood pressures is restricted, the minimum blood pressures occurring within the inflow cannula 108 are detectable. Therefore, the controller 103 can determine the pumping state directly from the minimum value of the pulsatile blood pressure occurring within the inflow cannula 108. The negative peaks of the graph shown in FIG. 8 allow the controller 108 to determine correct, over or under-pumping. An elevated minimum pressure is generally indicative of under-pumping, whilst a relatively low or negative minimum pressure is indicative of over-pumping. The correct or desired pumping state is wherein the minimum pressure is approximately 0 mmHg.

The controller 103 uses the detected pumping state to amend the speed set-point of the implantable blood pump 104 and in turn reduces the effect of the adverse pumping state.

The above descriptions describe only some of the embodiments of the present invention. Modifications may be obvious to those skilled in the art and may be made without departing from the scope and spirit of the present invention.

1. An implantable device, including: a cuff positioned to contact the outer surface of a tubular body carrying blood; and at least one sensor which measures blood pressure encapsulated within said cuff.

2. The device of claim 1, wherein said device does not occlude or adversely affect the flow of blood or blood pressure within a patient's circulatory system.

3. The device of claim 1, wherein said device includes at least two sensors and said sensors are aligned axially in respect to said tubular body.

4. The device of claim 1, wherein said device includes at least two sensors and said sensors are aligned radially in respect to said tubular body.

5. The device of claim 1, wherein said cuff is integrally formed within a cannula.

6. The device of claim 1, wherein said device is connected to a controller that determines the pumping state of said heart from changes in said pressure.

7. The device of claim 1, wherein said cuff comprises: silicone, velour or Dacron™.

8. The device of claim 6, wherein said device cooperates with a blood pump.

9. The device of claim 8, wherein said blood pressure is used in a feed back mechanism to control the pumping speed of said blood pump.

10. A system for controlling an implantable blood pump including: an implantable blood pump in fluid communication with a circulatory system to assist heart function; at least one inlet pressure sensor for measuring pressure of blood flow in an inlet of said implantable blood pump; a controller operatively connected to said inlet pressure sensor and said implantable blood pump; and said controller estimates the current pumping state of the heart from minimum of said pressure over a period of time and adjusts the speed of said implantable blood pump based on said current pumping state.

11. The system of claim 10, wherein said inlet pressure sensor is encapsulated within a cuff adapted to contact the outer surface of a tubular body carrying blood.

12. The system of claim 10, wherein said period of time includes at least one cardiac cycle.

13. The system of claim 10, wherein said inlet pressure sensor detects a limited range near to the minimum of said pressure over a period of time.

14. The system of claim 10, wherein said inlet pressure sensor operates in a range between +50 and −50 mmHg.

15. The system of claim 10, wherein said controller adjusts pumping speed to minimise under-pumping and over-pumping by the implantable blood pump.

16. The system of claim 10, wherein said controller calculates blood flow from back EMF generated by the implantable blood pump, when in use.
